(19) United States
(12) Patent Application Publication
(10) Pub. No.: US 2005/0232911 A1
(43) Pub. Date: Oct. 20, 2005

(54) Prevention and treatment of metabolic abnormalities associated with excess intramyocellular lipid

(76) Inventor: Brian D. Schreiber, Neenah, WI (US)

Correspondence Address: WILHELM LAW SERVICE, S.C., 100 W LAWRENCE ST, THIRD FLOOR, APPLETON, WI, 54911, US

(21) Appl. No.: 11/109,148

(22) Filed: Apr. 19, 2005

Related U.S. Application Data
(60) Provisional application No. 60/563,430, filed on Apr. 19, 2004.

(51) Int. Cl. A61K 38/43; A61K 31/555; A61K 31/426; A61K 31/202; A61K 31/155; A61K 31/17; A61K 31/385

(52) U.S. Cl. 424/94.1; 514/184; 514/369; 514/440; 514/595; 514/635; 514/560

(57) ABSTRACT

Compositions and methods for treating and/or preventing metabolic syndrome in a subject exhibiting clinical and/or biochemical manifestations of metabolic syndrome, or being predisposed to developing metabolic syndrome, or a precursor indicator for metabolic syndrome. The method comprises administering a pharmaceutically-effective form of carnitine or a pharmaceutically acceptable salt thereof, alone or in combination with another agent, optionally in sustained-release format, in an amount effective to treat at least one symptom of metabolic syndrome or a precursor indicator for metabolic syndrome.

### BACKGROUND

The present invention relates to methods of preventing and/or treating precursor risk factors, of pre-disposition to various undesired health conditions including clogged arteries, cardiovascular disease, diabetes, stroke, kidney disease, or pancreatic malfunction, as well as the metabolic syndrome risk factor. In particular, the present invention relates to compositions and methods for preventing and/or treating such precursor risk factors, including metabolic syndrome, and resulting complications.

Metabolic syndrome is recognized under numerous designations including, but not limited to, plurimetabolic syndrome, dysmetabolic syndrome, syndrome X, insulin resistance syndrome, the deadly quartet, and Reaven's syndrome. Metabolic syndrome is most prevalent in the adult population, but can occur in children and adolescents, as well. It is estimated that as many as twenty-percent or more of adults in the United States of America have metabolic syndrome.

Individuals who have metabolic syndrome are at a relatively greater risk of developing certain undesired health conditions, as compared to healthy/normal individuals. Certain of these undesired health conditions are potentially life-threatening, including but not limited to, diabetes, cardiovascular disease, and stroke.

Metabolic syndrome is characterized as an assemblage of numerous risk factors. Accordingly, clinical criteria for diagnosis of the metabolic syndrome typically include the presence/expression of multiple ones of the risk factors.

Exemplary risk factors, which characterize metabolic syndrome, include obesity (particularly abdominal obesity), hypertension, blood abnormalities and/or disorders, insulin resistance, glucose intolerance, and others.

Not all risk factors need be present in an individual for the individual to be diagnosed as having metabolic syndrome. Also, the magnitude/severity of expression of individual ones of the risk factors may be relatively greater or lesser than the magnitude/severity of expression other ones of the risk factors. Which risk factors are present and/or expressed, and the respective magnitude of expression of each or any risk factor, can vary greatly between individual persons having metabolic syndrome.

Central obesity, e.g. abdominal obesity, can be characterized as relatively higher amounts of adipose tissue being disposed at an individual's trunk, than at the individual's limbs. The presence of relatively high amounts of adipose tissue can correspond to relatively high blood plasma levels of nonesterfied fatty acids.

Elevated plasma levels of nonesterfied fatty acids can in turn increase the amount of lipid in organ tissues such as muscle and liver. Obesity, in general, can contribute to cardiovascular disease, stroke, and Type II Diabetes.

Hypertension, e.g. blood pressure of, for example, ≧140/≧90 mm Hg increases the risk of coronary heart disease which can lead to myocardial infarction, and increases the risk of stroke.

Blood abnormalities and/or disorders include, but are not limited to, blood lipid disorders, which can be associated with myocardial infarction and stroke.

Specifically, blood lipid disorders can lead to plaque buildups in arteries, and/or arterial wall degradational changes.

Atherogenic dyslipidemia (the lipid triad), a blood lipid disorder, can be characterized by hypertriglyeridemia (high levels of triglycerides), high levels of LDL cholesterol, and low levels of HDL cholesterol, and can eventually compromise the integrity of arterial walls.

Insulin resistance is characterized as a state in which a typical amount of insulin produced by the pancreas, and resident in the blood stream, fails to interact normally with cells, making it more difficult for glucose to pass from the blood stream into e.g. muscle cells, liver cells, and adipose tissue. Given the onset of insulin resistance, the pancreas, over time, produces increased amounts of insulin and the increased insulin level in the blood stream helps pass glucose from the blood stream into the muscle cells, liver cells and adipose tissue. Increasing pancreatic insulin production can be a burden on, and can eventually become associated with pancreatic exhaustion manifested by a reduced ability of the pancreas to secrete insulin.

Individuals who have insulin resistance sometimes have glucose intolerance. Glucose intolerance is characterized as blood sugar levels which are greater than normal levels, yet less than diabetic levels. One example of glucose intolerance is impaired fasting glucose, e.g. fasting plasma glucose between 110 and 125 mg/dl (6.1 to 6.9 mmol/L). Another expression of glucose intolerance is fasting plasma glucose levels higher than normoglycemic levels, normoglycemic levels being defined as fasting plasma glucose levels of <110 mg/dl (6.1 mmol/liter).

Additional risk factors for metabolic syndrome can include abnormal blood levels of fibrinogen, plasminogen activator inhibitor −1, and/or C-reactive protein.

Abnormal levels of these compounds can lead to a prothrombotic state and/or a proinflammatory state, which may be related to strokes.

Certain lifestyle modifications such as weight loss and increased exercise are beneficial to subjects having metabolic syndrome, as well as subjects having frank diabetes and/or cardiovascular disease. Unfortunately, adoption of such changes by patients, in practice, is often limited and difficult to achieve.

Present pharmacological therapies have either been directed toward patients who have frank diabetes or such therapies have had side effect profiles, such as weight gain, which have provided difficulties in application of such therapies to the pre-diabetic population, including the population which has metabolic syndrome and other precursor risk factors of predisposition to stroke, diabetes, or cardiovascular disease.

Metabolic syndrome can be an indicator of predisposition of developing frank Type II Diabetes. Type II Diabetes Mellitus is an increasingly important cause of morbidity and mortality in both developed and emerging societies. The evolution of Type II Diabetes typically progresses from an early stage of insulin resistance through impaired glucose tolerance and, finally, the clinical and biochemical indices of frank diabetes, e.g. fasting plasma glucose levels at or above 126 mg/dl (7.0 mmol/L) or a random plasma glucose level (or 2 hour value in an oral glucose tolerance test) at or above 200 mg/dl (11.1 mmol/L).

Whereas frank diabetes is well known to be associated with major morbidities including retinopathy, neuropathy and cardiovascular disease, pre-frank diabetes conditions such as metabolic syndrome, insulin resistance and impaired glucose tolerance have, as well, been more recently associated with increased cardiovascular risk.

Specifically, insulin resistance even in the absence of frank diabetes has been shown to be an independent predictor of myocardial infarction and/or stroke. Considering the large number of patients in these early stages of disease, such as for example the large number of patients who have metabolic syndrome; this associated risk represents a major threat to individual and public health.

The evolution to Type II Diabetes is characterized not only by abnormalities in glucose metabolism, but also by various abnormalities in lipid metabolism as well. These include elevations in circulating free fatty acids as well as deposition of excess fat in muscle cells.

A tight negative relationship has been demonstrated between whole body insulin sensitivity and intramyocellular lipid (IMCL) in patients who have normal glucose tolerance as well as in patients who have impaired glucose tolerance and Type II Diabetes Mellitus; intramyocellular lipid being a lipid moiety which is located within the muscle cell as opposed to being located in the interstices between muscle cells. The distinction between the quantity of intramyocellular lipid and the quantity of intermyocellular lipid, namely the lipid which is located in the interstices between the muscle cells, can be determined by muscle biopsy and magnetic resonance testing, such as magnetic resonance spectroscopy, or other examination.

The relationship between impaired insulin sensitivity and intramyocellular lipid accumulation in the evolution of diabetes in the obese Zucker fatty rat, an accepted animal model of Type II diabetes mellitus, has been demonstrated. The insulin resistant, but not yet diabetic, offspring, of Type II diabetic patients having a significant increase in intramyocellular lipid in the soleus and tibialis muscle compared with their non insulin resistant siblings, has been demonstrated.

A possible mechanism by which excess intramyocellular lipid impairs insulin mediated glucose disposal has been suggested wherein the glucose transport mechanism is one site of inhibition by free fatty acids (FFA). The metabolism of one of the principal long chain fatty acids, namely palmitate, was altered in the muscle of insulin resistant obese Zucker rats.

Despite an increase in fractional and total uptake of palmitate into the cell in the obese compared with the lean Zucker rat, the percentage of palmitate oxidized was not proportionately increased so that a greater amount was stored in the cell as triglyceride.

Magnetic resonance spectroscopy has been used to measure intramyocellular lipid in the obese Zucker rat, with the resultant finding that there was a distinct relation between IMCL and insulin resistance.

Moreover, when rats were treated with roziglitazone so as to reduce the concentration of IMCL, there was a marked improvement in insulin sensitivity. That the same physiology prevails in humans is suggested by a study in which a related compound, troglitazone, was administered to patients at high risk for diabetes and was associated with prevention of frank Type II Diabetes.

Unfortunately, troglitazone has been removed from the market due to excessive hepatotoxicity, and related congeners such as roziglitazone have side effects such as weight gain that have limited their use in a population in whom excess obesity is already a significant issue.

A likely causal relationship between accumulation of intramyocellular lipid and insulin resistance, has been recognized. One specific intramyocellular lipid moiety that has been suggested as being of particular importance in stimulating insulin resistance is long chain acyl CoA. Acyl CoA is a chemical moiety comprised of a fatty acid derivative bound to the molecule Coenzyme A.

Significantly elevated basal long chain acyl CoA in the red quadriceps of high fat fed insulin resistant rats as well as a failure of hyperinsulinemia to cause a reduction in these levels similar to what is seen with insulin treatment of chow fed insulin sensitive controls has been demonstrated.

Indeed, at basal insulin levels, total long chain acyl CoA levels in red quadriceps muscle in high fat fed insulin resistant rats was increased by more than 200%, over levels seen in insulin sensitive chow fed animals. Moreover, long chain acyl CoA has been shown to exert substantial regulatory control over a number of enzymes involved in glucose metabolism.

Long chain acyl CoA may also promote the enhanced hepatic glucogenesis observed in fat fed insulin resistant rats via an inhibition of glucokinase. Additional mechanisms of action of long chain acyl CoA contributing to insulin resistance have been suggested to include stimulation and inhibition of various protein kinase C subtypes, promotion of complex lipid formation, regulation of genes encoding critical metabolic enzymes or transduction factors, and effects on protein acylation.

The fact that it is the accumulation of intramyocellular acyl CoA moieties per se rather than the impaired oxidation of these compounds that is responsible for insulin resistance was supported by studies which showed that the combination of high fat feeding and carnitine palmitoyl transferase I inhibition was a more potent suppressor of insulin stimulated glucose disposal than was either factor alone.

Additional studies suggest that the specific lipid moiety associated with reduction of insulin mediated glucose sensitivity is long chain acyl CoA. In a study which used both lard feedings and Etomoxir to increase LCACoA in whole animal models, both interventions were associated with a rise in intramyocellular lipid as well as increasing degrees of whole body insulin resistance.

Levocarnitine is an endogenous compound essential for the metabolism of LCACoA both through promotion of fatty acid oxidation and promotion of export of unmetabolized acyl moieties from the inner aspect of the mitochondrion. Oral levocarnitine is indicated in the treatment of primary systemic carnitine deficiency as well as for the acute and chronic treatment of an inborn error of metabolism which results in a secondary carnitine deficiency.

The administration of levocarnitine to patients with discrete disorders of fatty acid oxidation is frequently followed by excretion of the respective unmetabolized acyl moiety as acylcarnitines in the urine. The ability of levocarnitine to elute long chain acyl CoA esters has been demonstrated in canine cardiac tissue.

Studies have shown the ability of carnitine treatment to promote an increased urinary excretion of long chain carnitine esters presumably derived from precursor long chain acyl CoA. An additional reduction of long chain acyl CoA could result from the combined action of CPT-1 and CPT-2 to lower the long chain acyl CoA concentration in the mitochondria by eluting excess acetyl groups from the mitochondrion as acetylcarnitine. Thus the precursor to malonyl CoA may be reduced, presumably resulting in decreased intracellular lipogenesis.

Applicant contemplates that excess intramitochondrial long chain acyl CoA and an increased acetyl/free CoA ratio promote insulin resistance, and thus the elution of such moieties will enhance insulin sensitivity.

Moreover, acute administration of levocarnitine to Type II Diabetics has resulted in significantly increased whole body glucose uptake, and both glucose storage and oxidation, namely increased insulin sensitivity. Whereas an acute improvement in insulin sensitivity is intriguing, it is of limited clinical relevance.

On the other hand, were chronic administration of levocarnitine demonstrated to decrease the concentration of intramyocellular lipid in subjects having metabolic syndrome or other precursor risk factors of clogged arteries, cardiovascular disease, diabetes, stroke, kidney disease, or pancreatic malfunction, especially those disposed to diabetes, the physiology described above is compatible with the possibility of improving insulin sensitivity on a chronic basis in those whose sensitivity has been impaired and, indeed avoiding or postponing the onset of frank diabetes in those predisposed by virtue of either family history of diabetes, possessing the significant risk factor of obesity, or possessing other risk factors associated with metabolic syndrome or other precursor risk factors.

There is thus a need to provide a treatment for individuals who exhibit clinical and/or biochemical manifestations of metabolic syndrome or other precursor risk factors. In addition there is a need to provide a method of preventing metabolic syndrome for individuals, especially those individuals who are predisposed to developing metabolic syndrome.

Accordingly, there is a need to provide a method of treating metabolic syndrome or other risk factors by evaluating clinical and/or biochemical indications of metabolic syndrome or other risk factors and administering a medicament based at least in part on such evaluation.

There is a further need to provide a method of treating metabolic syndrome and/or other risk factors by administering a medicament which delays the onset of metabolic syndrome.

There is also a need to provide a method of diminishing the rate of progression of metabolic syndrome or reversing one or more of the risk factors related to metabolic syndrome.

There is another need to provide a method of treating metabolic syndrome and/or other risk factors by evaluating risk factors associated with metabolic syndrome and administering a medicament based at least in part on such evaluation.

### SUMMARY

This invention is directed toward improved compositions and methods for preventing, treating, and/or delaying the onset of certain disorders, associated with excess accumulation of intramyocellular lipid especially metabolic syndrome, and preventing, treating, and/or delaying the onset of certain diseases wherein metabolic syndrome or other precursor indications can be early indicators of increased risk of onset of such diseases. More specifically, the invention provides carnitine-based compositions, and associated methods, for treating, preventing and/or delaying the onset of metabolic syndrome, and diseases wherein metabolic syndrome and other precursor indicators can be early indications of increased risk of onset of such diseases. In some embodiments, the invention provides methods of evaluating individuals who have metabolic syndrome or other precursor risk factors for pre-disposition to clogged arteries, cardiovascular disease, diabetes, stroke, kidney disease, or pancreatic malfunction, and/or individuals who may be predisposed to developing metabolic syndrome, and methods of treating such individuals.

In a first family of embodiments, in a method of treating or preventing metabolic syndrome in a human exhibiting clinical and/or biochemical manifestations of metabolic syndrome, or being predisposed to developing metabolic syndrome, comprises administering carnitine or a pharmaceutically acceptable salt thereof in an amount effective to treat at least one symptom of metabolic syndrome.

In some embodiments, the method further comprises administering carnitine or a pharmaceutically acceptable salt thereof in an amount effective to stabilize and/or improve insulin sensitivity in such human.

In some embodiments, the method further comprises administering carnitine or a pharmaceutically acceptable salt thereof in an amount effective to prevent and/or delay the onset of insulin resistance in such human.

In some embodiments, the method further comprises administering carnitine or a pharmaceutically acceptable salt thereof in an amount effective to stabilize and/or improve glucose tolerance in such human.

In some embodiments, the method further comprises administering carnitine or a pharmaceutically acceptable salt thereof in an amount effective to prevent and/or delay the onset of glucose intolerance in such human.

In some embodiments, the method further comprises administering carnitine or a pharmaceutically acceptable salt thereof in an amount effective to stabilize and/or improve hyperglycemia in such human.

In some embodiments, the method further comprises administering carnitine or a pharmaceutically acceptable salt thereof in an amount effective to prevent and/or delay the onset of hyperglycemia in such human.

In some embodiments, the method further comprises administering carnitine or a pharmaceutically acceptable salt thereof in an amount effective to stabilize and/or reduce intramyocellular lipid content in such human.

In some embodiments, the method further comprises administering carnitine in a plurality of dispensations, ones of the dispensations being temporally spaced from other ones of the dispensations by generally regular temporal intervals in such human.

In some embodiments, the method further comprises the carnitine administered as an oral formulation.

In some embodiments, the method further comprises a carrier adapted to carry the carnitine in at least one of a tablet, capsule, pill, liquid, syrup, gel, slurry, powder, or granulated preparation.

In some embodiments, the method further comprises the carnitine being administered as a parenteral formulation.

In some embodiments, the method further comprises the parenteral formulation being administered by at least one of a subcutaneous, intramuscular, or intravenous route.

In some embodiments, the method further comprises administering a second agent.

In some embodiments, the method further comprises administering carnitine and the second agent, is performed in sequence.

In some embodiments, the method further comprises administering carnitine and the second agent is performed generally simultaneously.

In some embodiments, the method further comprises wherein carnitine and the second agent are part of a single medicament.

In some embodiments, the method further comprises the second agent effective in treating non-insulin dependent diabetes mellitus.

In some embodiments, the method further comprises the second agent a hypoglycemic agent and/or insulin sensitizing agent.

In some embodiments, the method further comprises the second agent being selected from the group consisting of biguanides, thiazolidinediones, sulfonylureas, meglitinides, alpha-glucosidase inhibitors, lipase inhibitors, and metglitinide analogs.

In some embodiments, the method further comprises the second agent being pioglitazone.

In some embodiments, the method further comprises the second agent being metformin.

In some embodiments, the method further comprises the second agent being a biologically active agent selected from the group consisting of protein kinase C inhibitors, choline, lipoic acid, coenzyme Q, chromium compounds.

In some embodiments, the method further comprises the second agent being a macromolecule selected from the group consisting of essential fatty acids, polyunsaturated fatty acids, fish oil derivatives, cytokines, binders, fillers, preservatives, stabilizing agents, emulsifiers, buffers, and conjugated linoleic acid.

In some embodiments, the method further comprises the human being an adult.

In some embodiments, the method further comprises the human being an adolescent or child.

In a second family of embodiments, the invention comprehends a method of diagnosing and treating metabolic syndrome in a human comprising evaluating clinical and/or biochemical manifestations of metabolic syndrome and administering carnitine or a pharmaceutically acceptable salt thereof based, at least in part, on such evaluation.

In some embodiments, the method further comprises evaluation including evaluating at least one risk factor.

In some embodiments, the method further comprises the at least one risk factor includes ethnicity.

In some embodiments, the method further comprises the at least one risk factor includes sibling expression of symptoms of frank diabetes.

In some embodiments, the method further comprises the at least one risk factor includes parental expression of symptoms of frank diabetes.

In some embodiments, the method further comprises the at least one risk factor includes obesity in such human.

In some embodiments, the method further comprises the at least one risk factor includes abnormal blood pressure in such human.

In some embodiments, the method further comprises the at least one risk factor includes atherogenic dylipidemia in such human.

In some embodiments, the method further comprises the at least one risk factor includes elevated blood levels of at least one of fibrinogen, plasminogen activator inhibitor −1, and C-reactive protein in such human.

In some embodiments, the method further comprises the at least one risk factor includes such human having experienced prolonged states of insufficient exercise.

In a third family of embodiments, the invention comprehends a method of evaluating predisposition of a human to metabolic syndrome and preventing metabolic syndrome or delaying the onset of metabolic syndrome in such human, the method comprising evaluating at least one risk factor and, based at least in part on such evaluation, administering carnitine or a pharmaceutically acceptable salt thereof in an amount effective to prevent and/or delay the onset of at least one symptom of metabolic syndrome.

In a fourth family of embodiments, the invention comprehends a method of diminishing the rate of progression of metabolic syndrome in a human comprising evaluating clinical and/or biochemical manifestations of metabolic syndrome and/or evaluating predisposition for clinical and/or biochemical manifestations of metabolic syndrome, and administering carnitine or a pharmaceutically acceptable salt thereof in an amount effective to diminish the rate of progression of metabolic syndrome based, at least in part, on such evaluation.

In a fifth family of embodiments, the invention comprehends a method of reducing the magnitude of expression of clinical and/or biochemical manifestations of metabolic syndrome in a human comprising evaluating clinical and/or biochemical manifestations of metabolic syndrome and administering carnitine as sustained-release carnitine or a pharmaceutically acceptable salt thereof in an amount effective to attenuate the expression of the clinical and/or biochemical manifestations of metabolic syndrome, whereby such carnitine is slowly released while in the gastrointestinal tract of such human.

In a sixth family of embodiments, the invention comprehends a method for preventing and/or treating metabolic syndrome in a subject comprising the coordinated administration of at least a first agent and second agent, the first agent comprising sustained-release carnitine.

In a seventh family of embodiments, the invention comprehends a composition for preventing and/or treating metabolic syndrome in a subject comprising a first agent and at least a second agent, the first agent comprising sustained-release carnitine.

In some embodiments the second agent being effective in treating non-insulin dependent diabetes mellitus.

In some embodiments the second agent comprises a hypoglycemic agent and/or insulin sensitizing agent.

In some embodiments the second agent selected from the group consisting of biguanides, thiazolidinediones, sulfonylureas, meglitinides, alpha-glucosidase inhibitors, lipase inhibitors, and metglitinide analogs.

In some embodiments the thiazolidinedione (second agent) is pioglitazone.

In some embodiments the thiazolidinedione (second agent) is roziglitazone.

In some embodiments the second agent is metformin.

In some embodiments the second agent is selected from the group consisting of protein kinase C inhibitors, choline, lipoic acid, coenzyme Q, chromium compounds

In some embodiments the second agent is selected from the group consisting of essential fatty acids, polyunsaturated fatty acids, fish oil derivatives, cytokines, binders, fillers, preservatives, stabilizing agents, emulsifiers, buffers, and conjugated linoleic acid.

In some embodiments the composition further comprises a third agent selected from the group consisting of protein kinase C inhibitors, choline, lipoic acid, coenzyme Q, chromium compounds.

In some embodiments a third agent selected from the group consisting of essential fatty acids, polyunsaturated fatty acids, fish oil derivatives, cytokines, binders, fillers, preservatives, stabilizing agents, emulsifiers, buffers, and conjugated linoleic acid.

In some embodiments the first agent comprises at least one of a non-eroding matrix delivery system, a hydrophilic eroding matrix delivery system, a coated matrix delivery system, and an osmotic based delivery system.

In some embodiments the subject is an adult human.

In some embodiments the subject is an adolescent or child human.

In some embodiments the subject is a mammal.

In an eight family of embodiments, the invention comprehends a composition for preventing and/or treating metabolic syndrome in a subject comprising at least a second agent and first agent, the first agent comprising sustained-release carnitine, and the second agent comprising immediate-release carnitine, the first agent and the second agent being in general communication with each other.

In some embodiments the first agent comprises at least one of a non-eroding matrix delivery system, a hydrophilic eroding matrix delivery system, a coated matrix delivery system, and an osmotic based delivery system.

In some embodiments the first agent comprises a semi-permeable layer.

In some embodiments the semi-permeable layer having at least one opening formed therethrough.

In some embodiments the second agent generally surrounds the first agent.

In a ninth family of embodiments, the invention comprehends a composition for preventing and/or treating metabolic syndrome in a subject comprising at least a second agent and first agent, the first agent comprising carnitine, and the second agent being effective in treating non-insulin dependent diabetes mellitus.

### BRIEF DESCRIPTION OF THE DRAWINGS

FIG. 1 shows a chemical structure of a levocarnitine molecule.

The invention is not limited in its application to the details of construction or the arrangement of the components set forth in the following description or illustrated in the drawings. The invention is capable of other embodiments or of being practiced or carried out in other various ways. Also, it is to be understood that the terminology and phraseology employed herein is for purpose of description and illustration and should not be regarded as limiting. Like reference numerals are used to indicate like components.

### DESCRIPTION OF THE ILLUSTRATED EMBODIMENTS

The present invention relates to methods of preventing, delaying the onset of, and/or treating metabolic syndrome and other indicators of lipid-related disorders. In particular, the present invention relates to methods of clinically treating and/or managing metabolic syndrome, and its precursor indicators, and correspondingly preventing and/or treating conditions, such as conditions/diseases which metabolic syndrome can be an early indicator of propensity towards, e.g. diseases which metabolic syndrome can be an early warning indicator of, such as diabetes mellitus type II, with preparations comprising Levocarnitine (L-carnitine), its pharmacologically acceptable salts and/or its precursors, alone or in combination with other agents and/or modifiers or control substances.

The present invention relates to early and advanced stages in the progression of metabolic syndrome, as well as to precursor indicators of metabolic syndrome. In particular, the present invention relates to treatment/prevention of symptoms of metabolic syndrome such as impaired glucose tolerance, insulin resistance, hyperglycemia, and others, in subjects who exhibit clinical and/or biochemical manifestations of, or who are at elevated risk for development of, such symptoms. The invention further relates to diagnosis and treatment of precursor symptoms of insulin resistance in subjects having normal insulin sensitivity.

As used herein, the term insulin resistance, as a symptom of metabolic syndrome, is a state in which a given concentration of insulin is associated with a subnormal glucose response. Insulin resistance is a component of a number of states which include, but are not limited to, the following:
extreme insulin resistance syndromes such as the type B syndrome which have autoantibodies associated with the insulin receptor, leprechaunism with insulin receptor mutations and the lipodystrophic states;
insulin resistance associated with intake of pharmacologic protean inhibitor drugs, valproic acid, olanzapine, clozapine and psychoactive agents;
insulin resistance associated with obesity, stress, infection, acromegaly, Cushings Syndrome or Disease, the polycystic ovary syndrome, ovarian hyperthecosis;
insulin resistance unassociated with other pathological states;
resistance to endogenous insulin as defined by a high serum insulin concentration in association with blood glucose concentrations that are normal or high;
resistance to exogenous insulin as evident in patients treated with either insulin or the oral hypoglycemic agents and/or insulin sensitizing agents described herein who require either abnormally high doses of insulin or the specified oral hypoglycemic agent and/or insulin sensitizing agent to prevent or treat hyperglycemia.

Insulin resistance can be defined as an abnormal value according to e.g. generally known and/or accepted methods/procedures of determining insulin resistance and/or sensitivity. Those skilled in the art are well aware of certain methods/procedures of determining such insulin resistance and/or sensitivity values, including but not limited to, the homeostasis model assessment-insulin resistance (HOMA-IR), whole body insulin sensitivity index (WBISI), insulin sensitivity index (ISI), euglycemic-hyperinsulinemic clamp measure, and others.

Homeostasis model assessment-insulin resistance (HOMA-IR) can be calculated using the equation: (HOMA-IR)=(FI×FG)/22.5, wherein FI is the fasting insulin concentration (in microunits per milliliter) and FG is the fasting glucose lever (in millimoles per liter). Accordingly, relatively lower HOMA-IR values correspond to relatively greater insulin sensitivity, whereas relatively higher HOMA-IR values correspond to relatively lower insulin sensitivity.

Whole body insulin sensitivity index (WBISI) can be calculated using parameters obtained from a standard oral glucose tolerance test (OGTT) (1.75 g/kg body weight (up to 75 g)), and using the equation: WBISI=10,000/square root of [(fasting glucose×fasting insulin)×(mean (OGTT) glucose×mean (OGTT) insulin)].

Insulin sensitivity index (ISI) can be calculated using parameters obtained from a standard oral glucose tolerance test (OGTT) (1.75 g/kg body weight (up to 75 g)), and using the equation: ISI_{OGTT}=[1.9/6×body weight (kg)×fasting plasma glucose (mmol/liter)+520−1.9/18×body weight×area under the glucose curve (mmol/h·liter)−urinary glucose (mmol)/1.8]/[area under the insulin curve (pmol/h·liter)×body weight].

Euglycemic-hyperinsulinemic clamp measure is traditionally used to determine an “M-value of insulin sensitivity” as can be determined by the equation: “M-value”=INF−UC−SC, wherein INF is the necessary infusion rate (in milligrams per meter squared per minute) of glucose to maintain the euglycemic clamp, UC is the correction for urinary loss of glucose, and SC is the space constant. The space constant adjusts for the changes in glucose concentration during the clamp.

Patients who have insulin resistance as a symptom of metabolic syndrome may exhibit either normal or abnormal blood glucose levels, and also may have normal or only slightly high blood glucose concentrations. This group includes patients with obesity including general obesity, visceral obesity, and centripetal obesity, increased waist/hip ratio; as well as patients with high serum free fatty acid, serum cholesterol and/or triglyceride, lipid profile that includes hypertriglyceridemia and low serum high density lipoprotein concentration; hypertension, including hypertension even with minimal elevations; and including impaired glucose tolerance, hyperandrogenism, and those with inherited syndromes of severe insulin resistance such as the Type A syndrome.

Subjects at risk for insulin resistance include those who have components/symptoms of metabolic syndrome including, but not limited to, absolute, central or visceral obesity, abnormally high waist/hip ratio, hypertriglyceridemia with low HDL, subjects receiving HAART (highly active antiretroviral therapy including protease inhibitors), subjects with polycystic ovary disease or other hyperandrogenism.

Also at risk are subjects who have Cushings disease or subjects ingesting exogenous corticosteroids, subjects taking growth hormone or having acromegaly, as well as subjects taking valproic acid, olanzepine, clozapine or lamotrigine.

Also at risk are subjects who have at least one of (i) relatively elevated levels of protein kinase C isoforms, e.g. elevated levels of active protein kinase C isoforms, (ii) impaired and/or relatively low rates of insulin-mediated tyrosine phosphorylation of insulin receptor substrate-1, (iii) impaired and/or relatively low rates of insulin receptor substrate-1 associated with phosphatidylinositol 3-kinase activity, (iv) and relatively elevated concentrations of long chain acyl-CoA in muscle cells.

Some subjects are genetically predisposed to developing metabolic syndrome and/or symptoms of metabolic syndrome, such as insulin resistance. Subjects who are genetically predisposed include children where one or both parents have diabetes mellitus, or members of certain ethnic groups, such as Native Americans, Hispanics or Aboriginal groups who have altered their diet to that of a more Western type e.g. high caloric density, high fat and animal products. In addition, metabolic syndrome can be associated with subjects who have experienced prolonged periods of time wherein they lacked sufficient exercise.

Clinical diagnosis of metabolic syndrome and/or recognition/evaluation of symptoms and/or risk factors of metabolic syndrome can be done based, at least in part, on the following risk factors. In the invention, such diagnosis may be followed by treatment of metabolic syndrome by administering pharmaceutical formulations which comprise L-carnitine and/or its analogues and/or precursors, alone or in combination with at least one other agent.

Metabolic syndrome has been defined by different study groups in terms of different sets of risk factors. For example, the ATP III panel of the National Cholesterol Education program based its clinical diagnosis of metabolic syndrome, at least in part, on risk factors, including
abdominal obesity defined by waist circumference
   in males as >102 cm (>40 inches) and
   in females as >88 cm (>35 inches);
triglycerides ≧150 mg/dl;
HDL cholesterol
   in males as <40 mg/dl (1.04 mmol/L) and
   in females as <50 mg/dl (1.29 mmol/L);
blood pressure being ≧130/≧85 mm Hg; and
fasting glucose being ≧110 mg/dl.

According to the WHO Criteria for Metabolic Syndrome, clinical diagnosis of metabolic syndrome is based, at least in part, on risk factors including
Type II Diabetes;
impaired fasting glucose levels;
impaired glucose tolerance;
glucose uptake below the lowest quartile for the background population under investigation under hyperinsulinemic, euglycemic conditions;
high blood pressure, e.g. ≧140 mm Hg systolic or ≧90 mm Hg diastolic, or antihypertensive mediation;
plasma triglycerides ≧150 mg/dl (≧1.7 mmol/L);
HDL cholesterol
   in males as <35 mg/dl (<0.9 mmol/L) and
   in females <39 mg/dl (<1.0 mmol/L);
body mass index (BMI)>30 kg/m²;
waist to hip ratio
   in males as >0.9 and
   in females as >0.85;
urinary albumin excretion rate ≧20 μg/min;
albumin to creatine ratio ≧30 mg/g.

According to the American Association of Clinical Endocrinologists, clinical diagnosis of metabolic syndrome is based, at least in part, on risk factors including
body mass index (BMI)>25 kg/m²;
elevated triglycerides triglycerides ≧150 mg/dl (≧1.7 mmol/L);
low HDL cholesterol
   in males as <40 mg/dl (<1.04 mmol/L) and
   in females <50 mg/dl (<1.29 mmol/L);
elevated blood pressure being ≧135 Hg systolic or ≧85 mm Hg.
2-hour postglucose challenge >140 mg/dl;
fasting glucose between 110 mg/dl and 126 mg/dl;
cardiovascular disease (CVD);
polycystic ovary syndrome;
sedentary lifestyle;
advancing age;
family history of Type II Diabetes;
family history of cardiovascular disease (CVD);
ethnicity having high risk of Type II Diabetes; and
ethnicity having high risk of cardiovascular disease (CVD).

As used herein with respect to the scope of the invention, including the scope of the claims, the definition of metabolic syndrome can include, in general, any of the clinical diagnosis definitions of ATP III, of the WHO, or of the American Association of Clinical Endocrinologists. However, the definition of metabolic syndrome, when addressing the scope of the invention, including the claims, does not, under any circumstances, include type II diabetes, namely does not include fasting glucose levels >126 mg/dl.

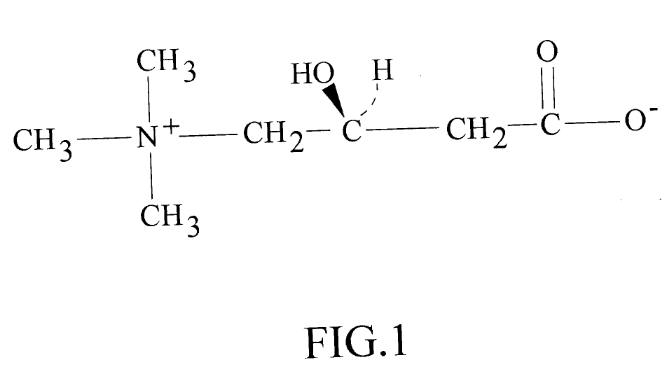

The carnitine, e.g. 3-hydroxy-4-N trimethylammonium butyrate, as in FIG. 1, analog can be a precursor of any of the forms of carnitine. Precursors of carnitine include, but are not limited to, gamma-butyrobetaine, lysine, and methionine. In some embodiments, precursors of carnitine include cofactors which enable carnitine to be endogenously manufactured from such precursors, in the body. Such cofactors include, but are not limited to, niacin (B3), thiamin (B1), B6, Vitamin C, and iron.

In some embodiments, the carnitine analog can be a pharmaceutically acceptable salt of carnitine. In some embodiments, the 3-hydroxy-4-N trimethylammonium butyrate (carnitine) analog is acetyl-L-carnitine or propionyl-L-carnitine or alkanoyl-L-carnitine. In other embodiments, the carnitine analog is acetyl-L-carnitine hydrochloride, propionyl-L-carnitine hydrochloride, L-carnitine hydrochloride, L-carnitine tartrate, L-carnitine fumarate, or L-carnitine magnesium citrate.

In general, the present invention favorably modifies insulin resistance in a subject whose insulin resistance is abnormally elevated or is predictably at elevated risk of becoming elevated. In some embodiments, the present invention lowers blood or plasma glucose levels in subjects whose fasting or postprandial glucose is elevated beyond a normal or desirable range.

In some embodiments, the present invention favorably modifies and/or prevents insulin resistance by favorably modifying at least one of (i) levels of protein kinase C isoforms, e.g. levels of active protein kinase C isoforms, (ii) rates of insulin-mediated tyrosine phosphorylation of insulin receptor substrate-1, (iii) rates of insulin receptor substrate-1 associated with phosphatidylinositol 3-kinase activity, (iv) and concentrations of long chain acyl-CoA in muscle cells.

Those skilled in the art are well aware of apparatus and methods suitable for quantifying e.g. levels of protein kinase C isoforms and/or the mRNA of protein kinase C isoforms, including but not limited to proteonomics, polymerase chain reactions, gene chip analysis, enzymatic assays, and other well known and future well known suitable apparatus and methods.

The present invention can prevent or inhibit or delay the progression to euglycemic insulin resistance, from euglycemic insulin resistance to impaired glucose tolerance, or can prevent or inhibit or delay the progression from impaired glucose tolerance to frank diabetes mellitus.

The subject having metabolic syndrome and insulin resistance or impaired glucose tolerance can be a human, or can be e.g. a rodent or other non-human mammal or other animal. The human or animal subject can be immature, or can be an adult animal or human.

The present invention provides for administration of a pharmaceutical drug formulation of levocarnitine or its acetylcarnitine or propionylcarnitine congeners, or other forms of carnitine, or precursors of carnitine, as noted herein, as an agent. In some embodiments, the pharmaceutical drug formulation includes a formulation designated as a dietary supplement of or containing levocarnitine, acetylcarnitine, propionylcarnitine as an agent, or other carnitine salts, such as those having a carnitine inner salt, e.g. carnitine fumarate, as an agent.

The L-carnitine can be administered in conjunction with another agent. For example, the L-carnitine and the other agent are administered in sequence, e.g. the administration of L-carnitine and the other agent are temporally separated from each other. In other embodiments, the L-carnitine and the other agent are administered generally simultaneously. In yet other embodiments, the L-carnitine and the other agent are administered as parts of a single medicament.

The 3-hydroxy-4-N-trimethylammonium butyrate analog can be combined in a pharmaceutical drug formulation with a hypoglycemic agent (as another agent) and/or insulin sensitizing agent (as another agent) including members of the following classes: biguanides (including metformin), thiazolidinediones, sulfonylureas, meglitinides, alpha-glucosidase inhibitors, lipase inhibitors, and metglitinide analogs.

The present invention can include combination of L-carnitine in a drug formulation with another agent which comprises at least one of, but not limited to, the following agents: A protein kinase C inhibitor for synergy in the treatment, prevention, or delay of onset of metabolic syndrome; specifically the symptoms of insulin resistance, impaired glucose tolerance or future disease complications such as frank Type II Diabetes and the complications thereof. In some embodiments the carnitine moiety is combined with an agent such as an essential or nonessential fatty acid including but not limited to an N3 fatty acid, fish oil derivative, or conjugated linoleic acid (CLA).

The 3-hydroxy-4-N trimethylammonium butyrate (L-carnitine) analog can be given as an oral formulation or as a parenteral formulation. The pharmaceutical compositions of the present invention can be formulated using pharmaceutically acceptable carriers well known in the art in dosages suitable for oral administration.

Such carriers enable the pharmaceutical compositions to be formulated as tablets, capsules, pills, liquids, syrups, gels, slurries, powder or granulated preparations, suspensions and the like, as well as any other known form, for oral or nasal ingestion by a patient to be treated, alternatively known forms suitable for parenteral administration, e.g. forms suitable for subcutaneous, intramuscular, or intravenous administration.

In light of various criteria related to metabolic syndrome, and its precursor indicators, as desired, multiple administrations per day of L-carnitine are provided to the user. And in some instances, in light of various criteria related to metabolic syndrome, and its precursor indicators, L-carnitine is otherwise made bioavailable to the user for a relatively greater temporal period, per day, as compared to e.g. a single administration of an immediate-release L-carnitine medicament. Such relatively greater temporal period of bioavailable exposure to the user can be achieved by various means which include, but are not limited to, administration by various sustained-release delivery systems and/or compositions.

Thus, it is contemplated that to treat and/or managing metabolic syndrome, and its precursor indicators, an oral formulation of any of the above might be given in, for example, a sustained-release composition, such that the active ingredients therein are released from the administered medicament, thereby to enter the bloodstream, over a period of at least about 2 hours, alternatively at least about 4 hours.

Pharmaceutical compositions of the invention, utilizing sustained-release compositions and/or structures, might incorporate any of the suitable sustained-release delivery systems, which enable an active ingredient to be released over a period of time. Such sustained-release delivery systems include, but are not limited to, matrix systems, osmotic based delivery systems, and others.

Matrix based systems of sustained-release delivery include non-eroding matrix delivery systems, hydrophilic eroding matrix delivery systems, and coated matrix delivery systems. Pharmaceutical compositions, utilizing non-eroding matrices as sustained-release systems, include systems having the agent dissolved in, embedded in, or dispersed in, a matrix of another material that delays release of the agent and/or agents into, e.g. the gastrointestinal tract of a recipient.

Non-eroding matrix sustained-release systems include but are not limited to non-eroding hydrogel matrices and non-eroding matrix tablets. Non-eroding matrix compositions can be manufactured by various methods of manufacturing non-eroding matrix delivery systems, well known to those skilled in the art, such as extrusion/spheroidization, rotary granulation, and other processes.

Hydrophilic matrix sustained-release systems include, but are not limited to, a hydrophilic matrix tablet comprising the agent and/or agents, and a hydrophilic polymer enabling delayed release of the agent and/or agents into e.g. the gastrointestinal tract of a recipient. Hydrophilic matrix compositions can be manufactured by various means of manufacturing hydrophilic matrix delivery systems, well known to those skilled in the art, such as extrusion/spheroidization, rotary granulation, wet granulation, and other processes.

Coated matrix delivery systems can comprise a tablet, which includes the agent and/or agents, which is coated by a generally impermeable coating having an aperture, or other opening, formed therethrough. Coated matrix delivery systems can be manufactured by conventional means of manufacturing coated matrix delivery systems; well known to those skilled in the art.

Such methods of manufacturing can include coating part of the surface of a tablet, which comprises the agent or agents, with a generally impermeable material/coating. Other methods of manufacturing can include coating a tablet, which comprises the agent or agents, and mechanically or otherwise forming an aperture or opening through such coating, e.g. drilling, punching, cutting, and others.

Osmotic based delivery systems include but are not limited to, delivery systems sometimes referred to as “osmotic pumps.” Osmotic pump sustained-release delivery systems comprise a core which includes the agent and/or agents in an osmotically effective composition. The core is surrounded by a semi-permeable coating, e.g. a coating which permits water to pass through the coating in a first direction while generally preventing solutes from passing through the coating in a second, opposite direction. In general at least one aperture and/or opening extends through the coating to the core.

Thus, in an osmotic pump sustained-release pharmaceutical compound, water enters the delivery system through the aperture, and thus enters the core. Once the water is in the core, the agent and/or agents become dissolved in the water, which in turn builds hydrostatic pressure in the pharmaceutical compound at the core. As the hydrostatic pressure in the pharmaceutical compound increases at the core, the osmotic pressure gradient across the semi-permeable coating correspondingly increases. When the hydrostatic pressure and/or osmotic pressure gradient increases to a sufficiently high level, the agent and/or agents are forced, e.g. pumped out of the osmotic system through the at least one aperture and/or opening formed through the semi-permeable membrane, optionally through the semi-permeable membrane itself.

As desired to treat and/or managing metabolic syndrome, and its precursor indicators, an oral formulation can be given whereby part of the dose, e.g. some of the pharmaceutically-active agent and/or agents, is immediately released from the administered medicament, and part of the dose, e.g. some of the pharmaceutically-active agent and/or agents, is released in a sustained-release manner, such that a part of the dose of active ingredient/agent and/or agents therein enter the bloodstream both generally immediately, and over a period of at least about 2 hours, alternatively at least about 4 hours.

To treat and/or managing metabolic syndrome, and its precursor indicators, the pharmaceutical compositions can comprise a first, immediate-release component and a second, sustained release component. Accordingly, in ome embodiments, the pharmaceutical composition is formulated to release a portion of the agent and/or agents shortly after administration, while another portion of the agent and/or agents is released in a sustained-release manner over a prolonged period of time.

The pharmaceutical composition can comprise a multiple-layered medicament. One layer of the medicament contains the agent and/or agents in an immediate-release form, e.g. a tablet layer comprising the agent and/or agents, absent a sustained-release component. A second, separate layer of the medicament contains the agent and/or agents in a sustained release form, e.g. a tablet layer comprising the agent and/or agent as part of at least one of a non-eroding matrix delivery system, hydrophilic eroding matrix delivery system, coated matrix delivery system, or osmotic based delivery system, or other sustained release form. Methods of making a bi-layer tablet suitable for use in the present invention are well known to those skilled in the art.

The medicament can comprise an immediate-release coating layer, which generally surrounds/coats a sustained-release core. The immediate-release coating layer is released shortly after administration, thereby leaving the sustained-release core to interact with, e.g. the gastrointestinal tract of a recipient. Methods of making such a medicament are well know those skilled in the art, and include using coating equipment, conventional in the pharmaceutical industry, to coat an immediate-release drug coating, which comprises the agent and/or agents, over a medicament core comprising the agent and/or agents in at least one of e.g. a non-eroding matrix delivery system, hydrophilic eroding matrix delivery system, coated matrix delivery system, and osmotic based delivery system. Matrix materials, suitable for fabricating sustained-released components, include pharmacologically used and known polymers, which are well known to those skilled in the art.

Pharmaceutical compositions suitable for use in the present invention include compositions wherein the active ingredients are contained in an effective amount to achieve the intended purpose. The active ingredients include L-carnitine, its analogs (pharmaceutically acceptable salts) and precursors, hypoglycemic agent and/or insulin sensitizing agent including, but not limited to members of the following classes: biguanides (including metformin), thiazolidinediones, sulfonylureas, meglitinides, alpha-glucosidase inhibitors, lipase inhibitors, and metglitinide analogs; biologically active agents including, but not limited to protein kinase C inhibitors, choline, lipoic acid, coenzyme Q, chromium compounds; essential fatty acids, polyunsaturated fatty acids, fish oil derivatives, and conjugated linoleic acid.

Other agents which may be incorporated into the medicament include cytokines, binders e.g. cellulose, cellulose esters, cellulose ethers, methyl cellulose, hydroxypropyl cellulose (DPC), hydroxypropyl methyl cellulose (HPMC), paraffin, carnauba wax, beeswax, modified vegetable oils, hydrogenated castor oil, polyethylene oxide, poly-N-vinyl-2-pyrrolidinone, polyvinyl alcohol, polyvinyl acetate, polyvinyl chloride, ethylene vinyl acetate, polystyrene, xanthan gum, carrageenan, preservatives, stabilizing agents, emulsifiers, buffers, and others.

An effective amount of L-carnitine and its analogues is that amount which decreases insulin resistance and/or decreases hyperglycemic blood glucose levels and/or prevents the emergence or worsening of insulin resistance in patients who have, or are at predictable elevated risk of developing, metabolic syndrome, or insulin resistance, or impaired glucose tolerance, or are at risk for diabetes mellitus type II.

As one example, a therapeutically effective dose refers to the amount of L-carnitine and/or its analogues which ameliorates, prevents or stabilizes increased insulin resistance or frank hyperglycemia, as a symptom of metabolic syndrome, in the situations outlined herein.

Normal doses of L-carnitine administered pursuant to the invention typically vary from about 100 mg up to a total dose of about 15,000 mg, e.g. 100 mg-15,000 mg, 250 mg-10,000 mg, and 500 mg-8,000 mg, depending upon the route of administration, the frequency of administration, and the time period over which the medicament is effectively released into the blood stream of the recipient thereof.

A typical dose of L-carnitine can approximate 50 mg per kg of body weight, ranging from about 5 mg per kg of body weight up to about 100 mg per kg of body weight.

As another example, an effective amount of other hypoglycemic agents and/or insulin sensitizing agents which may be combined with the carnitine is that amount which assists in decreasing insulin resistance and/or decreasing hyperglycemic blood glucose levels and/or preventing the emergence or worsening of insulin resistance in patients who have, or are at risk for, metabolic syndrome, or insulin resistance, or impaired glucose tolerance or diabetes mellitus type II.

A normal dose of such hypoglycemic agents and/or insulin sensitizing agents can vary based on variables including, but not limited to, the identity of the particular hypoglycemic agents and/or insulin sensitizing agents administered, route of administration, age of recipient, body weight of recipient, and others.

As one example, oral administration of pioglitazone to an adult corresponds to a dose approximating about 15 mg/day to about 45 mg/day. As another example, oral administration of metformin to an adult corresponds to a dose approximating about 1000 mg/day to about 2550 mg/day.

For any compound used in the methods of the invention, the therapeutically effective dosage can be formulated in animal models to achieve a desirable circulating concentration range. Toxicity and therapeutic efficacy of such compounds can be determined by standard pharmaceutical procedures in cell cultures or experimental animals, e.g., for determining LD50 (the lethal dose to 50% of the population) and the ED50 (the dose therapeutically effective in 50% of the population). The dose rated between toxic and therapeutic effects is the therapeutic index, expressed as the ratio LD50/ED50 of a compound or compounds, with large therapeutic indices being preferred.

The exact dosage is chosen by the individual physician in consideration of the patient to be treated, adjusting the dose and schedule of administration to provide sufficient levels of the active moiety to achieve and/or maintain the desired effect. Normal dosage amount of levocarnitine (L-carnitine) and its analogs varies from 100 mg to 15,000 mg per day, depending upon the route of administration, size and weight of the patient, renal and lepatic status, gastrointestinal sensitivity, and tolerance, age and gender of the patient.

### THE EXAMPLES

In-Vivo Experiments

General:

Forty-five male obese Zucker rats are obtained at 5 weeks of age (Charles is River) and randomized into three groups of 15 each. Namely, the forty-five male obese Zucker rats are randomized into Control Group A, Control Group B, and L-Carn Group, which each consists of 15 rats. Control Group A and Control Group B receive normal rat chow throughout the study. The 15 Zucker rats in L-Carn Group receive 100 mg/kg bodyweight (BWt) of levocarnitine, daily, in their drinking water or mixed with their food. At 6 weeks of age, all groups are placed in metabolic cages for 24-hours to determine baseline protein and creatinine excretion rates, whereby proteinuria can be determined. Systolic blood pressure measurements are obtained by tailcuff plethysmography and a sample of blood is obtained from the tail for determination of fasting plasma creatinine, insulin, blood glucose, triglycerides, and cholesterol. These measurements are subsequently repeated at 12, 18, and 24 weeks of age.

Insulin Sensitivity:

At 6 weeks of age, the Zucker rats of Control Group A are anesthetized with sodium pentobarbital (50-60 mg/kg BWt intraperitoneally) and insulin sensitivity is determined using the euglycemic-hyperinsulinemic clamp technique, explained elsewhere herein. The rats will fast overnight, and be anesthetized with sodium pentobarbital (50-60 mg/kg BWt, i.p.). Blood glucose is determined every 15 minutes over a 120 minute baseline period. Insulin is given as a bolus (48 mU/kg BWt×min) followed by a constant infusion at 4.8 mU/kg×min for an additional 120 minutes. A 15% solution of D-glucose is then infused into the femoral vein.

Blood glucose is analyzed every 5 minutes from a catheter in the femoral artery using a glucometer (Freestyle®, test volume required=0.3 μL) and the rate of D-glucose infused is adjusted to maintain plasma glucose at ˜5 mol/L. Whole body insulin sensitivity is calculated at the mean glucose infusion rate during the last 60 minutes of the euglycemic-hyperinsulinemic clamp study. Additional blood samples are taken at 120 minutes and 240 minutes and are collected from the femoral artery catheter into K-EDTA tubes. The additional samples are immediately centrifuged at 4° C., and plasma free fatty acid levels are determined within 60 minutes using a Wako Clinical Diagnostics spectrophotometric assay. In addition, some plasma is frozen at −80° C. for determination of insulin concentrations by Enzyme Immunometric Assay (EIA). Those skilled in the art are well aware of suitable EIA techniques and companies that perform such techniques, including American Laboratory Products Company (ALPCO Diagnostics, of Windham, N.H.).

Intramyocellular Lipid Content:

After determining insulin sensitivity, the adductor magnus muscle of the left hind leg is isolated and the anterior and posterior ends tied with 3-O silk and cut outside the ties. The muscle is placed across a wooden stick, slightly wider than the muscle section is long, and tied in place to preserve its normal amount of stretch, then immediately immersed in isopentane cooled in liquid nitrogen.

Serial transverse sections of tissue, having thicknesses of about 10 μm, are immediately cut on a Leica CM 1900 cryostat at −20° C. The serial transverse sections of tissue are each mounted on poly-L-lysine-coated slides.

The lipid content of the sections is determined by using histochemical staining. One such suitable histochemical staining process includes using lipid-soluble dye to dye respective sections of tissue which are then observed and/or other wise analyzed. A suitable such lipid-soluble dye is oil red O dye, preferably at a concentration of about 3 g/L in isopropanol.

Those skilled in the art are well aware of suitable preparation, observational, and analysis, techniques, using oil red O dye in 3 g/L isopropanol, to determine lipid content of tissue sections. As one example, the 10 μm tissue sections are stained by incubating the sections, mounted on the slides, at room temperature in the oil red O dye in 3 g/L isopropanol solution. The slides are then rinsed, whereby the resultant stained sections exhibit chromatically distinct portions which correspond to distinct substances within the mounted tissue.

Namely, various lipids, such as triglycerides, appear relatively orange-red in tint, and are thereby optically or otherwise distinguishable from other substances within the mounted tissue. Images are captured using an e.g. Photometrics CoolSnap CCD camera connected to a Nikon Diaphot inverted microscope and an e.g. Dell brand computer with Image ProPlus 4.5.1 image analysis software. The Image ProPlus 4.5.1 image analysis software enables calculation of cross-sectional area per muscle fiber, number of lipid droplets/unit area, mean diameter/droplet, and total lipid concentration (total area of droplets) as a percentage of the respective cross-sectional area.

Determination of Muscle Fiber Types (Types I, IIA, and IIB):

Serial transverse sections of tissue, adjacent those stained with oil-red-O, are stained with myofibrillar adenosine triphosphatase to determine the different muscle fiber types, e.g. type I, type IIA, and type IIB. Those skilled in the art are well aware of suitable staining techniques and methods of analysis, e.g. which include staining sections of tissue with myofibrillar adenosine triphosphatase, which enable the user to differentiate between various ones of the different fiber types based, at least in part, on the histochemical staining properties.

Ones of the three muscle fiber types are examined and lipid concentrations are determined respectively, pursuant to techniques and methods well known to those skilled in the art. Namely, determinations of number of lipid droplets/unit area, mean diameter/droplet, and total lipid concentration (total area of droplets) as a percentage of the respective cross-sectional area, are made.

Renal Histology:

After obtaining muscle tissue, the kidneys are perfusion fixed via the abdominal aorta with ˜100 ml of 1.25% glutaraldehyde in phosphate-buffered saline (PBS), for histology. Sections of kidney, which are about 1 mm in thickness, are embedded in paraffin, sectioned to about 3-4 μm in thickness, stained with periodic acid-Schiff (PAS), to enable determination of glomerular volume, glomerular sclerosis, mesangial expansion, and tubulointerstitial disease.

As one example, the PAS stained kidney sections are e.g. observed, examined, studied, and/or analyzed with light microscopy or other suitable means, while considering suitable observational criteria which includes, but is not limited to, a suitable glomerular sclerosis index, a suitable tubulointerstitial index, a suitable mesangial matrix index, and/or others. At 24 weeks of age, Control Group B and the L-Carn Group will be studied in a similar fashion with respect to general procedures, protocols, analyses, and techniques, as indicated in the Timeline chart, below.

Timeline:
AGE

| Group (n) | 5 weeks | 6 weeks | 12 weeks | 18 weeks | 24 weeks |
|---|---|---|---|---|---|
| Control A | Arrive | P, CCr, BP | | | |
| (12) | | IS, IMP, | | | |
| | | RH | | | |
| Control B | Arrive | P, CCr, BP | P, CCr, | P, CCr, | P, CCr, BP |
| (12) | | | BP | BP | IS, IMP, |
| | | | | | RH |
| Control C | Arrive | P, CCr, BP | P, CCr, | P, CCr, | P, CCr, BP |
| (12) | | Place on I- | BP | BP | IS, IMP, |
| | | carnitine | | | RH |

P = 24 h proteinuria, CCr = creatinine clearance, BP = systolic blood pressure by tailcuff plethysmography, IS = insulin sensitivity, IML = intramyocellular lipid content, RH = renal histology.

Analytical Techniques:

Urine and plasma creatinine, plasma total cholesterol, HDL cholesterol, LDL+VLDL cholesterol (taken as the difference between total and HDL cholesterol), triglyceride, and insulin levels are determined. Those skilled in the art are well aware of suitable methods of adequately determining all of such levels, concentrations, and/or other quantifications.

As one example, urine and plasma creatinine levels, are determined by methods including, but not limited to, various suitable pharmakinetic analyses, suitable colorometric techniques, and/or other suitable determinations, at least some of which are made using what those skilled in the art refer to as “Jaffe's reaction.”

Cholesterol level determinations, e.g. plasma total cholesterol, HDL cholesterol, LDL+VLDL cholesterol, are made, at least in part, using any of a variety of suitable cholesterol level determination kits, such as those available from Sigma-Aldrich Co. of St. Louis, Mo.

Triglyceride levels are determined, e.g. ezymatically, using various ones of suitable reagents and/or autoanalyzers.

Insulin levels are determined by any of a variety of suitable insulin level determining techniques and methods including, but not limited to, radioimmunoassays, insulin-specific assays, automated microparticle enzyme immunoassays, and others.

Plasma glucose concentrations are determined using ones of a variety of suitable techniques and methods. Exemplary of such suitable techniques and methods includes utilizing a Freestyle® system and analysis, which includes the use of suitable well known Freestyle® glucose monitors, meters, and/or other apparatus. In the alternative, plasma concentrations can be determined using various ones of a variety of suitable glucose testing kits, such as various glucose (Trinder) kits, available from Sigma-Aldrich Co. of St. Louis, Mo.

Plasma free fatty acid levels are determined by any of a variety of plasma free fatty acid level determining techniques and methods including, but not limited to, various chemical titration methods, thermometric titration methods, measurements of metal-fatty acid complexes, enzymatic methods, and various methods which use fatty acid binding proteins.

Statistical Analysis:

Statistical computations are performed using software under the trade name SigmaStat™ 2.03, from SPSS Inc., Chicago, Ill. Multiple group comparisons are tested using analysis of variance (ANOVA), each followed by the Tukey Test, for parameters with a normal distribution and homogeneity of variance. Data which is not normally distributed is log transformed and either analyzed by parametric tests, or analyzed by the Kruskal-Wallis one-way analysis of variance on ranks using Dunn's method for multiple comparisons.

Parameters measured over time (protein excretion, creatinine clearance, etc.) are analyzed using one-way repeated measures analysis of variance. Parameters measured over time but not normally distributed are analyzed using the Friedman repeated measures ANOVA on ranks, followed by the Dunn's Method. Categorical data, e.g. the glomerulosclerosis index, is analyzed using non-parametric techniques. Linear regression is used to determine the significance of correlations between measured parameters. Statistical significance is accepted when p<0.05.

Power Analysis for Determination of Animal Numbers:

Power analysis indicates the need for 12-13 successful experiments per group to achieve a power >0.80 with alpha=0.05 for one of the important parameters of interest, e.g. intramyocellular lipid concentration, based on the standard deviation expected from data, relating to intramyocellular lipid levels, obtained by others in lean and obese subjects and corresponding minimum detectable differences which are anticipated. Accordingly, 15 rats per group are used to generally ensure 12-13 successful experiments per group.

Results

The experiments show that chronic administration of levocarnitine decreases the intramyocellular lipid concentration in obese Zucker rats with metabolic syndrome and improves insulin sensitivity in these animals. A secondary benefit is the reduction of kidney injury in these animals because of the beneficial effects on insulin sensitivity, resulting in lower systemic insulin concentrations.

Similar examples can be given with any pharmaceutically acceptable formulation of levocarnitine, with or without a second pharmaceutically-active agent, in sustained-release form, or non-sustained release form.

Another method of diagnosing and treating pre-cursor indications of elevated risk of clogged arteries, cardiovascular disease, diabetes, stroke, kidney disease, or pancreatic malfunction, is to conduct a magnetic resonance test, for example a magnetic resonance spectroscopy test on muscle tissue of the subject, thus to examine the quantity of intramyocellular lipids (IMCL) in the muscle tissue. Where elevated levels of intramyocellular lipids are observed, such observation can be perceived as a precursor indicator of a condition presenting an elevated risk of metabolic syndrome, clogged arteries, cardiovascular disease, diabetes, stroke, kidney disease, or pancreatic malfunction, even though the subject may not be exhibiting any of the commonly-recognized clinical or biochemical expressions of such conditions.

Indeed, the invention comprehends administering magnetic resonance tests to a population of subjects, however selected and/or pre-screened, and on the basis of the magnetic resonance tests alone, administering an L-carnitine-containing medicament to those subjects who exhibit elevated intramyocellular lipid levels, above a threshold level, based solely on the results of the magnetic resonance tests. Such magnetic resonance test can be especially instructive when administered to subjects who do not exhibit any of the conventionally-recognized non-hereditary, non-environmental risk factors for e.g. stroke, diabetes, or cardiovascular disease, and can be used, according to the invention, as the only test on which to base a decision to administer L-carnitine-containing medicaments. Such test can, of course, also be used, as one of a number of indicators of such precursor condition for which L-carnitine treatment is desirable according to the invention, whereby the magnetic resonance test can be used as the first indicator or as an additional indicator, of desirability of treatment, with other tests known to be instructive in affirming the condition, being used to confirm, supplement, the magnetic resonance findings before the L-carnitine treatment is commenced.

Where conventionally-known hereditary or risk factors are present, the magnetic resonance test can be used as a treatment-triggering test where elevated intramyocellular lipids are detected. By using the magnetic resonance test as a treatment-triggering test, or by using the magnetic resonance test alone as a treatment-triggering test, effective intervention can be begun at a much earlier stage in development of clogged arteries, cardiovascular disease, diabetes, stroke, kidney disease, or pancreatic malfunction, namely before any clinical manifestations are present, and optionally before any other biochemical manifestations are present. In such case, treatment on the basis of the magnetic resonance spectroscopy tests alone, of intramyocellular lipids, can be effective in preventing and/or delaying onset of, or slowing down progression of, clinical manifestations and/or biochemical manifestations of clogged arteries, cardiovascular disease, diabetes, stroke, kidney disease, or pancreatic malfunction, as well as metabolic syndrome.

Typical magnetic resonance apparatus useful herein, such as to produce a magnetic resonance spectroscopy graph, is available as 1.5T Signa MR/i EchoSpeed Plus with M1033JD, Probe 2000, from GE Medical Systems, Waukesha, Wis.

Where elevated levels of IMCL are detected, and even though the subject does not exhibit indicators of metabolic syndrome risk factors, in the invention, the subject can be treated with a levocarnitine-containing medicament such as those disclosed herein, in suitable dosage to reverse or arrest, or slow down or delay further development of, the elevated levels of IMCL, or to slow down or delay the subsequent development of metabolic syndrome and/or clogged arteries, cardiovascular disease, diabetes, stroke, kidney disease, or pancreatic malfunction. Thus, the treatment is commenced before classic onset of metabolic syndrome, on the basis of elevated levels of intramyocellular lipids alone. Such early treatment with levocarnitine can slow down, or delay, or potentially prevent, the onset of metabolic syndrome, clogged arteries, cardiovascular disease, diabetes, stroke, kidney disease, or pancreatic malfunction.

The ¹H-MRS method to quantify intramyocellular lipid (IMCL) measures the relative intensity of the methylene (CH₂₎ₙ;1.25 part/million) resonance with that of water A normal level of IMCL by NMR spectroscopy is 2.21+/−0.11% of the water resonance peak intensity in adults whose mean age is 29+/−2 years. In children, insulin sensitivity has been seen to decrease when the % water resonance peak by magnetic resonance spectroscopy exceeds 1%. When an elevated level of IMCL has been detected, the actual level is compared to a threshold treatment level. Elevation of intramyocellular lipid has been shown to correlate with reduced insulin sensitivity as measured by the euglycemic-hyperinsulinemic clamp method, whole body insulin sensitivity index (WBISI) and the insulin sensitivity index (ISI). If the detected level is above the threshold level of 1% in a subject with other risk factors for insulin resistance as elaborated above or an absolute value of 2%, a suitable dosage of a levocarnitine-containing medicament is administered to the subject.

Suitable dosages of levocarnitine are those indicated earlier. The medicament can be levocarnitine alone, or levocarnitine in combination with another agent or agents. The medicament can be a sustained-release medicament of or a non-sustained release medicament.

The invention thus comprehends medicament compositions which optionally include levocarnitine in combination with other pharmaceutical agents wherein the collective affect of the combination is greater than the affect of levocarnitine alone. The medicament can be a sustained release medicament, or a non-sustained release medicament.

By using the magnetic resonance test as an indicator to begin the levocarnitine treatment, the recipient may avoid, or at least delay, development of insulin resistance or development of elevated glucose levels which are commonly-recognized indicators of metabolic syndrome. Thus, in some subjects, the development of metabolic syndrome can be delayed or avoided, or slowed down. The treatment intervention can thus be initiated at such early stage of development of blood glucose-related issues, e.g. elevated lipids in the cell, that the patient may be able to avoid or delay the demand for increased pancreatic production of insulin, thereby avoiding and/or delaying pancreatic failure, as well as the collection of negative health affects which are associated with insulin-related health defects.

Similarly, where the condition has progressed beyond the initial elevation of intramyocellular lipids to e.g. the development of metabolic syndrome, even though a more complex set of risk factors is in place, the negative metabolic affects of some or all of the risk factors may be controllable and/or reversible by administering L-carnitine-related medicaments as disclosed herein. Such early treatment can suppress and/or delay the further progression of development of such glucose-related diseases.

Accordingly, the invention provides effective intervention and treatment at an early stage in the development of defects related to the accumulation of intramyocellular lipid such as insulin resistance and elevated glucose levels. Indeed, the invention provides effective intervention and treatment in individuals wherein the standard known indicators of metabolic syndrome, or the standard precursor indicators of stroke, diabetes mellitus, or cardiovascular disease have not yet manifested or otherwise emerged—but where environmental and/or hereditary risk factors, and/or precursor indicators, are in place. Namely, where environmental and/or hereditary risk factors are in place, but the clinical or other biochemical manifestations are not yet exhibiting metabolic syndrome, the invention provides for initiating treatment on the basis of elevated intramyocellular lipids, as a method of reducing the level of intramyocellular lipids, and thereby delaying or arresting the development of metabolic syndrome and/or other indicators of clogged arteries, cardiovascular disease, diabetes, stroke, kidney disease, or pancreatic malfunction.

Those skilled in the art will now see that certain modifications can be made to the apparatus, compositions, combinations, and methods herein disclosed with respect to the illustrated embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

To the extent the following claims use means plus function language, it is not meant to include there, or in the instant specification, anything not structurally equivalent to what is shown in the embodiments disclosed in the specification.

1. A method of evaluating predisposition of a human to metabolic syndrome and preventing metabolic syndrome or delaying the onset of metabolic syndrome in such human, the method comprising evaluating at least one risk factor related to metabolic syndrome and, based at least in part on such evaluation, administering a pharmaceutically-effective form of carnitine or a pharmaceutically acceptable salt thereof in an amount effective to prevent and/or delay the onset of at least one symptom of metabolic syndrome.

2. A method as in claim 1 wherein the at least one risk factor includes ethnicity, or sibling expression of symptoms of frank diabetes, or parental expression of symptoms of frank diabetes, or obesity in such subject, or abnormal blood pressure in such subject, or atherogenic dyslipidemia in such subject, or elevated blood levels of at least one of fibrinogen, plasminogen activator inhibitor −1, and C-reactive protein in such subject, such subject having experienced prolonged states of insufficient exercise.

3. A method as in claim 1 wherein the subject is a human adult.

4. A method as in claim 1 wherein the subject is a human adolescent or child.

5. A method of diminishing the rate of progression of metabolic syndrome, or of diminishing manifestation of a precursor indicator for metabolic syndrome, in a subject, comprising evaluating clinical and/or biochemical manifestations of metabolic syndrome and/or evaluating predisposition for clinical and/or biochemical manifestations of metabolic syndrome, or manifestations of a precursor indicator for metabolic syndrome, and administering a pharmaceutically-effective form of carnitine or a pharmaceutically acceptable salt thereof in an amount effective to diminish the rate of progression of metabolic syndrome or the precursor for metabolic syndrome, based, at least in part, on such evaluation.

6. A method as in claim 5, further comprising administering the carnitine or pharmaceutically acceptable salt thereof in an amount effective to stabilize and/or improve insulin sensitivity in such subject or in an amount effective to reduce the rate of development of insulin insensitivity in such subject.

7. A method as in claim 5 further comprising administering the carnitine or pharmaceutically acceptable salt thereof in an amount effective to prevent and/or delay the onset of glucose intolerance in such subject.

8. A method as in claim 5 further comprising administering the carnitine or pharmaceutically acceptable salt thereof in an amount effective to stabilize and/or improve hyperglycemia in such subject.

9. A method as in claim 5 further comprising administering the carnitine or pharmaceutically acceptable salt thereof in an amount effective to stabilize and/or reduce intramyocellular lipid content in such subject.

10. A method as in claim 5 wherein the evaluation includes evaluating at least one risk factor.

11. A method as in claim 10 wherein the at least one risk factor includes ethnicity, or sibling expression of symptoms of frank diabetes, or parental expression of symptoms of frank diabetes, or obesity in such subject, or abnormal blood pressure in such subject, or atherogenic dyslipidemia in such subject, or elevated blood levels of at least one of fibrinogen, plasminogen activator inhibitor −1, and C-reactive protein in such subject, or such subject having experienced prolonged states of insufficient exercise.

12. A method as in claim 5, further comprising administering the carnitine or pharmaceutically acceptable salt thereof in an amount effective to prevent and/or delay the onset of insulin resistance in such subject.

13. A method as in claim 5 further comprising administering the carnitine or pharmaceutically acceptable salt thereof in an amount effective to stabilize and/or improve glucose tolerance in such subject.

14. A composition of matter for preventing and/or treating metabolic syndrome or a precursor to metabolic syndrome, in a subject, said composition of matter comprising a first agent, comprising a pharmaceutically-effective form of carnitine, and at least a second pharmaceutically-effective agent.

15. A composition as in claim 14, said second agent being effective in treating non-insulin dependent diabetes mellitus.

16. A composition as in claim 14, wherein said second agent comprises a hypoglycemic agent and/or an insulin sensitizing agent.

17. A composition as in claim 14, said second agent being selected from the group consisting of biguanides, thiazolidinediones, sulfonylureas, meglitinides, alpha-glucosidase inhibitors, lipase inhibitors, and metglitinide analogs.

18. A composition as in claim 17 wherein said second agent is selected from the group consisting of pioglitazone and metformin.

19. A composition as in claim 14 wherein said second agent is selected from the group consisting of protein kinase C inhibitors, choline, lipoic acid, coenzyme Q, and chromium compounds

20. A composition as in claim 14 wherein said second agent is selected from the group consisting of essential fatty acids, polyunsaturated fatty acids, fish oil derivatives, cytokines, binders, fillers, preservatives, stabilizing agents, emulsifiers, buffers, and conjugated linoleic acid.
